(19) United States
(12) Patent Application Publication
(10) Pub. No.: US 2002/0192169 A1
(43) Pub. Date: Dec. 19, 2002

(54) Matte-rendering cosmetic composition containing a polytetrafluoroethylene aqueous dispersion and use thereof

(75) Inventors: Veronique Chevalier, Villecresnes (FR); Melanie Quest, Paris (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 10/079,593

(22) Filed: Feb. 22, 2002

(30) Foreign Application Priority Data
Feb. 22, 2001 (FR) 01 02417

(51) Int. Cl. A61K 7/021; A61K 7/025

(52) U.S. Cl. 424/63; 424/64

(57) ABSTRACT

The invention concerns a matte-rendering cosmetic composition containing polytetrafluoroethylene (PTFE) in the form of a dispersion of particles in an aqueous medium, the particles having a numerical mean dimension ranging from about 0.1 to 30 μm, and preferably from 0.5 to 15 μm.

### BACKGROUND OF THE INVENTION

1. Field of the Invention

The present invention relates to a composition and its use in the cosmetic treatment of greasy skin. The composition is preferably a matte-rendering cosmetic composition, and is one containing, preferably in a physiologically acceptable medium, polytetrafluoroethylene (PTFE) in the form of a dispersion of particles in an aqueous medium.

2. Discussion of the Background

By definition, a matte-rendering product is a product which prevents the skin from glistening and which homogenizes the complexion. Skin care or make-up compositions having matte-rendering properties are usually used to resolve the problems of brilliance caused by excess sebum, and in order to improve the long-term appearance of make-up, since make-up has a tendency to deteriorate visually during the day. These compositions give a matte appearance to the skin, resulting from a capacity to diffuse light at the skin surface. They may also be used to soften skin defects such as microcontours, wrinkles, lines, pores or colour variations.

Generally, matte-rendering cosmetic compositions are formulated with the aid of coated or uncoated mineral fillers (ZnO, SiO_{2,}) and starch. These particles are very well known and very much used, but have the disadvantage of sedimenting. Hence there exists the need for a fluid matte-rendering cosmetic composition having a high viscosity threshold.

### SUMMARY OF THE INVENTION

The inventors have now found a composition overcoming the disadvantages of the prior art, and have made the surprising discovery that polytetrafluoroethylene (PTFE) in the form of a dispersion of submicronic particles in an aqueous medium confers, after application to the skin, a protracted matte appearance while being comfortable and non- dehydrating.

### DETAILED DESCRIPTION OF THE INVENTION

PTFE has been used in cosmetic compositions. International patent application WO/9103228 describes a cosmetic product containing a fibrillar polymer added to a compacted powder, where the fibrillar polymer can be polytetrafluoroethylene (PTFE) or polypropylene. Similarly, European patent application EP-A-0997134 describes the use of a particulate phase in a cosmetic composition to mask imperfections of the skin. This particulate phase contains polymeric-type particles of mean dimensions ranging from 5 to 100 μm. The particles may be constituted of polytetrafluoroethylene (PTFE). Finally, EP-A-0502769 describes matte-rendering compositions conferring a translucent layer and a natural appearance to the made-up skin. They are dispersions of spherical or spheroidal solid particles in a fatty binder, having dimensions ranging from 0.5 to 25 μm and constituted of a material compatible with a topical application of the composition, as for example PTFE. However, these references are silent concerning the matte-rendering effect contributed by aqueous dispersions of polytetrafluoroethylene (PTFE).

Hence, one subject of the present invention is the cosmetic use, for example for the treatment of greasy skin, of a matte-rendering cosmetic composition containing, preferably in a physiologically acceptable medium, a matte-rendering effective quantity of polytetrafluoroethylene (PTFE), wherein the polytetrafluoroethylene is in the form of a dispersion of particles in an aqueous medium having a mean numerical dimension ranging from about (±10%) 0.1 to 30 μm, and preferably from 0.5 to 15 μm.

A “matte-rendering composition” is a composition making it possible to render the appearance of the skin matte. By “matte appearance” of the skin a non-glistening appearance with homogeneous coloring is meant. “Physiologically acceptable medium” means a non-toxic medium capable of being applied to the skin (including the interior of the eyelids) or the lips of humans. An aqueous medium is both a medium containing only water and a medium containing water and a water-soluble solvent such as a lower alcohol containing from 1 to 6 carbon atoms or a glycol. Matte-rendering effective quantity means a quantity sufficient to attain the desired aim, i.e. a matte appearance. This quantity depends on the PTFE dispersion used and on other compounds of the composition, and can be determined by one of ordinary skill in the art in view of the guidance provided below.

The polytetrafluoroethylene (PTFE) used in the composition of the invention is not a film-forming polymer; this means that the PTFE does/would not form a film after evaporation of the water of the dispersion, and that the particles of copolymer remain at the surface of the support (in particular the skin) and diffuse light in all directions.

Polytetrafluoroethylene (PTFE) dispersions useful in the composition according to the invention include those sold under the names:

| | |
|---|---|
| 1. | X-6687 ™ (50% dispersion of polytetrafluoroethylene |
| | powder: size 0.7 μm) by the SHAMROCK Company, |
| 2. | Hydropure 9174C ™ (aqueous dispersion of |
| | polytetrafluoroethylene: size 3-4 μm) by the SHAMROCK |
| | Company |
| 3. | Fluotron 200 ™ (40% aqueous dispersion of |
| | polytetrafluoroethylene: size 12 μm) by the CARROLL |
| | SCIENTIFIC Company. |

PTFE is generally present in the invention composition in a quantity of active material (i.e., PTFE itself) ranging from 0.1 to 30% by weight, preferably from 0.15 to 15% by weight, and better still from 0.5 to 5% by weight of the total weight of the composition, including 1, 3, 7, 9, 11, 13, 17, 20, 23, 26 and 29% and all values and sub-ranges therebetween.

The composition may be provided in any form including the form of an aqueous composition, or in the form of a oil-in-water emulsion (O/W) or a water-in-oil emulsion (W/O), or a multiple emulsion. According to a preferred embodiment of the invention, it is provided in the form of an emulsion, in particular an oil-in-water emulsion (O/W) or a water-in-oil emulsion (W/O) or a multiple emulsion (W/O/W or O/W/O).

“Emulsion” in the sense of the application includes both dispersions without emulsifiers and dispersions containing emulsifiers, and also dispersions stabilized by solid particles or by tiny lipid spheres of the ionic or non-ionic type.

In the case of use for greasy skin, an oil-in-water (O/W) emulsion is preferred, the external aqueous phase of which also freshens the skin.

When the composition is an emulsion, it contains an oily phase.

The nature of the oily phase of the composition of the invention is not critical and may be constituted by all of the fat bodies, and in particular the oils, conventionally used in the cosmetics field. The oily phase usually contains at least one oil.

Oils useful in the composition of the invention include oils of plant origin (jujube, avocado, sesame, sunflower, maize, soy bean, saffron, grape seed), mineral oils (vaseline, isoparaffins optionally hydrogenated), synthetic oils (isopropyl myristate, cetearyl octanoate, polyisobutylene, ethyl-hexyl palmitate, alkyl benzoates), volatile and non-volatile silicone oils such as the polydimethylsiloxanes (PDMS) and the cyclodimethylsiloxanes or cyclomethicones, in particular cyclohexadimethylsiloxane and cyclopentadimethylsiloxane, and the fluorinated oils including fluorosiliconated oils, and their mixtures.

Other fat bodies which may be present in the oily phase include fatty acids, fatty alcohols such as stearyl alcohol, cetyl alcohol and cetearyl alcohol, the waxes and their mixtures.

In the compositions of the invention provided in the form of an emulsion, the aqueous phase of the composition may be present in a proportion of 1 to 80% by weight, and preferably in a proportion of 30 to 70% by weight of the total weight of the composition, and the oily phase may be present in a proportion of 5 to 70% by weight, and preferably in a proportion of 10 to 50% by weight of the total weight of the composition.

The emulsions may contain at least one emulsifier selected from the amphoteric, anionic, cationic or non-ionic emulsifiers, used alone or in a mixture. The emulsifiers are selected appropriately in accordance with the emulsion to be produced: water-in-oil (W/O) or oil-in-water (O/W) emulsions, which is within the skill of the ordinary artisan in view of this disclosure.

In the case of oil-in-water emulsions (O/W) useful emulsifiers include:

as amphoteric emulsifiers, the N-acyl-amino acids such as the N-alkylamino-acetates and the disodium cocoamphodiacetate and the amine oxides such as stearamine oxide;

as anionic emulsifiers, the acylglutamates such as “disodium hydrogenated tallow glutamate” (Amisoft HS-21™ sold by the AJINOMOTO company); the carboxylic acids and their salts such as sodium stearate; the phosphoric esters and their salts such as “DEA oleth-10 phosphate”; the sulfosuccinates such as “disodium PEG-5 citrate lauryl sulfosuccinates” and “disodium ricinoleamido MEA sulfosuccinate”;

as cationic emulsifiers, the alkyl-imidazolidinium salts such as isostearyl-ethylimidonium ethosulfate; ammonium salts such as N,N,N-trimethyl-1-docosanaminium chloride (behentrimonium chloride);

as non-ionic emulsifiers, the carbohydrate esters and ethers such as sucrose stearate, sucrose cocoate and the mixture of sorbitan stearate and sucrose cocoate sold by the ICI company under the name Arlatone 2121®;

polyol esters, particularly glycerol or sorbitol esters such as glyceryl stearate, polyglyceryl-2 stearate, sorbitan stearate ; glycerol ethers;

oxyethylenated and/or oxypropylenated ethers such as lauric alcohol oxyethylenated, oxypropylenated ether with 25 oxyethylenated groups and 25 oxypropylenated groups (CTFA name “PPG-25 laureth-25”) and the oxyethylenated ether of the C₁₂-C₁₅ fatty alcohol mixture containing 7 oxyethylenated groups (CTFA name “C₁₂-C₁₅ Pareth-7”) ; the polymers of ethylene glycol such as PEG-100.

It is possible to use one or more of these emulsifiers.

In the case of the water-in-oil (W/O) emulsions, useful emulsifiers include polyol fatty esters, in particular glycerol or sorbitol fatty esters, and in particular the polyol isostearic, oleic and ricinoleic esters, such as the petrolatum mixture of polyglyceryl-3 oleate, glyceryl isostearate, hydrogenated ricin oil and ozocerite, sold under the name Protegin W™ by the GOLDSCHMIDT Company, sorbitan isostearate, polyglyceryl di-isostearate, polyglyceryl-2 sequi-isostearate; carbohydrate esters and ethers such as “methyl glucose dioleate”; the fatty esters such as magnesium lanolate; the dimethicone copolyols and alkyl-dimethicone copolyols such as laurylmethicone copolyol sold under the name Dow Corning 5200 Formulation Aid™ by the DOW CORNING Company and the cetyl dimethicone copolyol sold under the name Abil EM 90™ by the GOLDSCHMIDT Company; and their mixtures.

The emulsifiers may be introduced as such or in the form of mixtures with other emulsifiers and/or with other compounds such as fatty alcohols or oils.

The composition of the invention may contain in addition conventional adjuvants such as water-soluble or liposoluble coloring matters, pigments, perfumes, preservatives, chemical or physical sun filters, chelators (EDTA), liposoluble or water-soluble active substances, hydrating agents such as polyols and in particular glycerol, pH adjusters (acids or bases) and fillers, used in their conventional amounts. These adjuvants preferably are present in quantities ranging from 0.01 to 20% by weight of the total weight of the composition. These adjuvants as well as their concentrations should be such that they do not modify the property desired for the composition.

As chemical sun filters usable in the composition of the invention, the composition of the invention may contain any of the UVA and UVB filters usable in the cosmetics field.

Useful UVB filters include:

1. the salicylic acid derivatives, in particular homomenthyl salicylate and octyl salicylate;

2. the cinnamic acid derivatives, in particular 2-ethylhexyl p-methoxycinnamate, sold by the Givaudan Company under the name Parsol MCX™;

3. the liquid β,β′-diphenylacrylate derivatives, in particular 2-ethylhexyl α-cyano-α,β′-diphenylacrylate or octocrylene, sold by the BASF Company under the name VINUL N539™;

4. the p-aminobenzoic acid derivatives;

5. 4-methyl benzylidene camphor sold by the Merck Company under the name EUSOLEX 6300™;

6. 2-phenylbenzimidazole 5-sulfonic acid sold under the name EUSOLEX 232™ by the Merck Company;

7. the 1,3,5-triazine derivatives, in particular:

2,4,6-tris [p-(2′-ethylhexyl-1′-oxycarbonyl) anilino]-1,3,5-triazine sold by the BASF Company under the name UVINUL T150™, and

dioctyl butamido triazone sold by the Sigma 3V Company under the name UVASORB HEB™;

8. mixtures of these filters.

Useful UVA filters include:

1. the dibenzoylmethane derivatives, in particular 4-(tert.-butyl) 4′-methoxy dibenzoylmethane sold by the Givaudan Company under the name PARSOL 1789™;

2. 1,4-[di(3-methylidenecampho-10-sulfonic]benzene acid, optionally in a partially or totally neutralized form, sold under the name MEXORYL SX™ by the Chimex Company;

3. the benzophenone derivatives, for example:

2,4-dihydroxybenzophenone (benzophenone-1),

2,2′,4,4′-tetra-hydroxybenzophenone (benzophenone-2),

2-hydroxy-4-methoxy-benzophenone (benzophenone-3), sold under the name UVINUL M40™ by the BASF Company,

2-hydroxy-4-methoxy-benzophenone-5 -sulfonic acid (benzophenone-4) as well as its sulfonate form (benzophenone-5), sold by the BASF Company under the name UVINUL MS40™,

2,2′-dihydroxy-4,4′-dimethoxy-benzophenone (benzophenone-6),

5-chloro-2-hydroxybenzophenone (benzophenone-7),

2,2′-dihydroxy-4-dimethoxy-benzophenone (benzophenone-8),

2,2′-dihydroxy-4,4′-dimethoxy-benzophenone-5,5′-disulfonic acid disodium salt (benzophenone-9),

2-hydroxy-4-methoxy-4′-methyl-benzophenone (benzophenone-10),

benzophenone-11,

2-hydroxy-4-(octyloxy) benzophenone (benzophenone-12);

4. the silane derivatives or benzophenone-containing polyorganosiloxanes;

5. the anthranilates, in particular menthyl anthranilate sold by the Haarman & Reiner Company under the name NEO HELIOPAN MA™;

6. the compounds bearing per molecule at least two benzoazolyl groups or at least one benzodiazolyl group, in particular 1,4-bis-benzimidazolyl-phenylene-3,3′,5,5′-tetrasulfonic acid as well as its salts sold by the Haarman & Reimer Company;

7. the silicon-containing derivatives of N-substituted benzimidazolyl-benzazoles or benzofuranyl-benzazoles, and in particular:

2-[1-[3-[1,3,3,3-tetramethyl-1-[(trimethylsilyl)oxy]disiloxanyl]-propyl]-1H-benzimidazol-2-yl]-benzoxazole,

2-[1-[3-[1,3,3,3-tetramethyl-1-[(trimethylsilyl)oxy]-disiloxanyl]-propyl]-1H-benzimidazol-2-yl]-benzothiazole

2-[1-(3-trimethylsilanyl-propyl)-1H-benzimidazol-2-yl]-benzoxazole,

6-methoxy-1,1′-bis-(3-trimethylsilanyl-propyl)-1H, 1′H-[2,2′]dibenzimidazolyl-benzoxazole,

2-[1-(3-trimethylsilanyl-propyl)-1H-benzimidazol-2-yl]-benzothiazole,

which are described in the patent application EP-A-1.028.120;

8. the triazine derivatives, and in particular 2,4-bis {[4-(2-ethylhexyloxy)-2-hydroxyl]-phenyl}-6-(4-methoxyphenyl)-1,3,5-triazine sold by the Ciba Geigy Company under the name TINOSORB S™, and 2,2′-methylenebis-[6-(2H benzotriazol-2-yl)-4-(1,1,3,3-tetramethylbutyl)phenol] sold by the Ciba Geigy Company under the name TINOSORB M™; and

9. their mixtures.

It is also possible to use a mixture of two or several of these filters and a mixture of UVB filters and UVA filters, and also mixtures with physical filters.

Useful physical filters include the oxides of titanium (titanium dioxide, amorphous or crystallized in the form of rutile and/or anatase), zinc, iron, zirconium, cerium or their mixtures. These metal oxides may be in the form of particles having a size in the micrometer or nanometer range (nanopigments). In the form of nanopigments, the mean particle sizes range for example from 5 to 100 nm. Nanopigments are preferably used in the composition of the invention.

The filters may be present in a quantity ranging from 0.01 to 20% by weight, and preferably from 0.1 to 10% by weight of the total weight of the composition.

Active substances that may be included in the composition of the invention include active substances useful for treating greasy skin such as zinc salts and in particular zinc gluconate, antibacterials such as vitamins B3 (niacinamide) and B5 (panthenol), zinc oxide and its derivatives, salicylic acid, triclosan, lipacid C8G® sold by the SEPPIC company or capryloylglycine, clove extract, octopirox, hexamidine, azelaic acid, anti-acne active substances and their mixtures.

Fillers useful in the composition according to the invention include silica powder, talc, polyamide particles and in particular those sold under the name ORGASOL™ by the ATOCHEM Company; polyethylene powders, acrylic copolymers-based microspheres such as those of the copolymer ethylene glycol dimethacrylate/lauryl methacrylate sold under the name POLYTRAP™ by the DOW CORNING Company; expanded powders, such as hollow microspheres and in particular the microspheres sold under the name EXPANCEL™ by the KERMANORD PLAST Company or the microspheres sold under the name MICROPEARL F 80 ED™ by the MATSUMOTO Company; powders of natural organic materials such as maize, wheat and rice starches, cross-linked or uncross-linked, such as the starch powders cross-linked by octenylglycosuccinic anhydride sold under the name DRY-FLO™ by the NATIONAL STARCH Company; silicone resin microbeads such as those sold under the name TOSPEARL™ by the TOSCHIBA SILICONE Company; and their mixtures. These fillers may be present in quantities ranging from 0 to 40% by weight, and preferably from 0.1 to 30% by weight, and better still from 0.5 to 10% by weight of the total weight of the composition.

Moreover, depending on the fluidity of the composition desired, it is possible to add one or more hydrophilic or lipophilic substances, selected for example from clays, polysaccharide gums and their derivatives (xanthan gum, carboxymethylcellulose, hydroxypropylguar), carboxyvinyl polymers or carbomers, the polyacrylamides such as that sold under the name SEPIGEL 305™ by the SEPPIC company, the at least partially cross-linked acrylamidomethylpropane sulfonic acid polymers such as the product sold under the name HOSTACERIN AMPS™ (CTFA name: ammonium polyacryldimethyltauramide) by the HOECHST Company, and their mixtures. These gelling agents may be used at concentrations ranging from 0.05 to 10%, preferably from 0.1 to 5% and better still from 0.1 to 3% of the total weight of the composition.

The composition according to the invention is preferably one adapted for topical application and finds in particular an application in a large number of cosmetic and/or dermatological treatments of the skin and in particular for the purpose of conferring on it a matte appearance.

The examples which follow serve to illustrate without in any way limiting the scope of the invention. The quantities are given in % by weight, unless indicated otherwise.

### EXAMPLE 1

Matte-rendering cream: O/W emulsion

| Oily phase: | | |
|---|---|---|
| Cyclohexadimethylsiloxane | | 10% |
| Stearyl alcohol | | 1% |
| Glyceryl stearate | | 2% |
| (and) PEG-100 stearate | | |
| Aqueous phase: | | |
| Xanthan gum | | 0.2% |
| Ammonium polyacryldimethyltauramide | | 0.4% |
| sold by the HOECHST company | | |
| under the name HOSTACERIN AMPS' | | |
| Sodium hydroxide | | 0.01% |
| Glycerine | | 5% |
| Aluminium starch octenylsuccinate | | 3% |
| sold by the NATIONAL STARCH company | | |
| under the name DRY-FLO: | | |
| PTFE in 40% aqueous dispersion | | 3.75% |
| (size: 12 μm) | | |
| sold by the CARROLL SCIENTIFIC Company | | |
| under the name FLUOTRON 200 | | |
| Preservative | | 0.70% |
| Water | qsp | 100% |

Preparation

The emulsion is prepared by adding, with shaking, the oily phase heated to 65° C. to the hot aqueous phase.

A composition is obtained suitable for rendering the skin matte.

### EXAMPLE 2

Matte-rendering cream: O/W emulsion

| Oily phase: | | |
|---|---|---|
| Cyclohexadimethylsiloxane | | 10% |
| Stearyl alcohol | | 1% |
| Glyceryl stearate | | 2% |
| (and) PEG-100 stearate | | |
| Aqueous phase: | | |
| Xanthan gum | | 0.2% |
| Ammonium polyacryldimethyltauramide | | 0.4% |
| sold by the HOECHST Company | | |
| under the name HOSTACERIN AMPS ™ | | |
| Sodium hydroxide | | 0.01% |
| Glycerine | | 5% |
| Aluminium starch octenylsuccinate | | 3% |
| sold by the NATIONAL STARCH Company | | |
| under the name DRY-FLO ™: | | |
| PTFE in 60% aqueous dispersion | | 2.5% |
| (size: 10 μm) | | |
| sold by the SHAMROCK Company | | |
| under the name HYDROPURE 9174C ™ | | |
| Preservatives | | 0.7% |
| Water | qsp | 100% |

The preparation of the emulsion is the same as that in Example 1.

A matte-rendering composition is obtained which eliminates the brilliance of the skin.

### COMPARATIVE EXAMPLE

Cream not containing PTFE: O/W emulsion

| Oily phase: | | |
|---|---|---|
| Cyclohexadimethylsiloxane | | 10% |
| Stearyl alcohol | | 1% |
| Mixture of dimerystyl tartrate/cetearyl alcohol/ | | 1.5% |
| C₁₂—C₁₅—Pareth-7/PPG-25 laureth-25 | | |
| sold by the ENICHEM Company | | |
| under the name COSMACOL PSE ™ | | |
| Aqueous phase: | | |
| Xanthan gum | | 0.2% |
| Ammonium polyacryldimethyltauramide | | 0.4% |
| sold by the HOECHST Company | | |
| under the name HOSTACERIEN AMPS ™ | | |
| Sodium hydroxide | | 0.01% |
| Glycerine | | 5% |
| Silica | | 1.5% |
| sold by the MIYOSHI Company | | |
| under the name SB 150 ™ | | |
| Aluminium starch octenylsuccinate | | 3% |
| sold by the NATIONAL STARCH Company | | |
| under the name DRY-FLO ™: | | |
| Preservatives | | 0.70% |
| Water | qsp | 100% |

The preparation of the emulsion is the same as that of Example 1.

A matte-rendering composition is obtained which eliminates the brilliance of the skin.

Test of Matteness

The matteness obtained for the compositions according to the Examples 1 and 2, each containing 1.5% of PTFE (as active material: AM), and for the comparison the comparative example contained 1.5% silica (AM) instead of PTFE, was measured.

The measurement was made in the following manner: the composition is spread on a rubber support at a concentration of 2 g/cm², it is left to dry, then the reflection was measured with the aid of a gonioreflectometer, the result obtained being the ratio R between the specular reflection and the diffuse reflection. The lower value of R, the greater the matte-rendering effect.

| | | | Comparative |
|---|---|---|---|
| Composition | Example 1 | Example 2 | example |
| R | 1.97 ± 0.07 | 1.87 ± 0.07 | 2.20 ± 0.08 |

These results in vitro show that with a PTFE concentration of 1.5% AM, a higher matteness is obtained than that obtained with the comparative formula which consists of 1.5% AM of silica.

### EXAMPLE 3

| Oily phase: | | |
|---|---|---|
| Cyclohexadimethylsiloxane | | 10% |
| Stearyl alcohol | | 1% |
| Glyceryl stearate | | 2% |
| (and) PEG-100 stearate | | |
| Aqueous phase: | | |
| Xanthan gum | | 0.2% |
| Ammonium polyacryldimethyltauramide | | 0.4% |
| sold by the HOECHST Company | | |
| under the name HOSTACERIN AMPS ™ | | |
| Sodium hydroxide | | 0.01% |
| Glycerine | | 5% |
| Aluminium starch octenylsuccinate | | 3% |
| sold by the NATIONAL STARCH Company | | |
| under the name DRY-FLO ™: | | |
| PTFE in 40% aqueous dispersion | | 3.75% |
| (size: 12 μm) | | |
| sold by the CARROLL SCIENTIFIC Company | | |
| under the name FLUOTRON 200 ™ | | |
| Mexoryl SX ™ | | 2% |
| Nanotitanium (nanometric titanium oxide) | | 2% |
| triethanolamine | | 0.4% |
| Preservatives | | 0.70% |
| Water | qsp | 100% |

The invention method is preferably applied to skin having a tendency to become greasy or greasy-looking, but can be applied to all skin to provide the above-described benefits of matte appearance and a homogeneous complexion.

One of ordinary skill is capable of using the invention composition as described above, for example by applying to the skin and/or lips 0.5-2 g of composition one or more times daily. While not limited to such application amounts or repetitions, one of ordinary skill can use this example as a guide in practicing the invention.

French patent application 0102417 filed Feb. 22, 2001 is incorporated herein by reference, as are all documents referred to above.

1. A method comprising applying to the skin or lips a matte-rendering effective amount of a composition comprising a dispersion of polytetrafluoroethylene particles in an aqueous medium, the particles having a mean numerical dimension ranging from about 0.1 to 30 μm.

2. The method according to claim 1, wherein the polytetrafluoroethylene particles are present in an amount ranging from 0.1 to 30% by weight based on the total weight of the composition.

3. The method according to claim 1, wherein said particles have a mean numerical dimension of 0.5 to 15 μm.

4. The method according to claim 1, wherein said composition is in the form of an oil-in-water (O/W) or water-in-oil (W/O) or multiple emulsion.

5. The method according to claim 4, wherein said emulsion comprises at least one oil selected from the group consisting of oils of plant origin, mineral oils, synthetic oils, silicone oils, and fluorinated oils.

6. The method according to claim 5, wherein said emulsion further comprises fat bodies selected from the group consisting of fatty acids, fatty alcohols and waxes.

7. The method of claim 4, wherein said emulsion comprises an aqueous phase and an oily phase, and wherein the aqueous phase represents from 1 to 80% by weight of the total weight of the composition, and the oily phase represents from 5 to 70% by weight of the total weight of the composition.

8. The method of claim 4, wherein said emulsion comprises an emulsifier selected from the group consisting of amphoteric, anionic, cationic, and non-ionic emulsifiers and mixtures thereof.

9. The method of claim 1, wherein said composition further comprises one or more adjuvants selected from the group consisting of water-soluble and liposoluble coloring matters, pigments, perfumes, preservatives, sun filters, liposoluble and water-soluble active substances, chelators, hydrating agents, pH adjusters and fillers.

10. The method of claim 1, wherein said composition further comprises a sun filter selected from the group consisting of UVA filters, UVB filters, physical filters and their mixtures.

11. The method of claim 1, wherein said composition further comprises an active substance useful for treating greasy skin selected from the group consisting of zinc salts, anti-bacterials, triclosan, lipacid, clove extract, octopirox, hexamidine, azelaic acid, anti-acne active substances, and their mixtures.

12. The method of claim 1, wherein said composition further comprises a filler selected from the group consisting of silica powder, talc, polyamide particles, polyethylene powders, acrylic copolymer-based microspheres, expanded powders, powders of natural organic materials, silicon resin microbeads, and their mixtures.

13. The method of claim 1, wherein said composition further comprises one or more hydrophilic or lipophilic gellants selected from the group consisting of clays, polysaccharide gums and their derivatives, carboxyvinyl polymers, polyacrylamides, at least partially cross-linked acrylamidomethylpropane sulfonic acid polymers, and their mixtures.

14. A composition comprising a dispersion of polytetrafluoroethylene particles in an aqueous medium, wherein said particles have a mean numerical dimension of from about 0.1 to 30 μm.

15. The composition according to claim 14, wherein the polytetrafluoroethylene particles are present in an amount ranging from 0.1 to 30% by weight based on the total weight of the composition.

16. The composition according to claim 14, wherein said particles have a mean numerical dimension of 0.5 to 15 μm.

17. The composition according to claim 14, wherein said composition is in the form of an oil-in-water (OW) or water-in-oil (W/O) or multiple emulsion.

18. The composition according to claim 17, wherein said emulsion comprises at least one oil selected from the group consisting of oils of plant origin, mineral oils, silicone oils, and fluorinated oils.

19. The composition of claim 17, wherein said emulsion comprises an aqueous phase and an oily phase, and wherein the aqueous phase represents from 2 to 80% by weight of the total weight of the composition, and the oily phase represents from 5 to 70% by weight of the total weight of the composition.

20. The composition of claim 14, wherein said composition further comprises one or more adjuvants selected from the group consisting of water-soluble and liposoluble coloring matters, pigments, perfumes, preservatives, sun filters, liposoluble and water-soluble active substances, chelators, hydrating agents, pH adjusters and fillers.
